(19) United States
(12) Patent Application Publication
(10) Pub. No.: US 2010/0204618 A1
(43) Pub. Date: Aug. 12, 2010

(54) ULTRASOUND EQUIPMENT FOR TREATING OF EDEMA AND USE THEREOF

(75) Inventors: Byoung-Hyun Min, Seoul (KR); So Ra Park, Seoul (KR); Soon Keun Kwon, Gyeonggi-do (KR)

Correspondence Address: LUCAS & MERCANTI, LLP, 475 PARK AVENUE SOUTH, 15TH FLOOR, NEW YORK, NY, 10016, US

(73) Assignee: REGENPRIME CO., LTD., Suwon-si, Gyeonggi-do (KR)

(21) Appl. No.: 12/671,075

(22) PCT Filed: Jul. 30, 2007
(86) PCT No.: PCT/KR2007/003653

§ 371 Date: Jan. 28, 2010

(51) Int. Cl. A61N 7/00

(52) U.S. Cl. 601/2

(57) ABSTRACT

The present invention relates to an ultrasound equipment for treating edema, and more particularly, to an ultrasound equipment to relieve symptoms of edema or repair cellular and tissue edemas by generating 20˜400 mW/cm² of low intensity ultrasound and use thereof.

The ultrasound equipment according to the present invention comprises a main control unit equipped with a control unit for ultrasound generation for generating a 20˜400 mW/cm² of low intensity ultrasound, an ultrasound generator for generating ultrasound according to the operating state of the main control unit, an ultrasound vibrator vibrating according to the value of ultrasound data inputted from the ultrasound generator, and a probe for delivering ultrasound energy to a treatment site, and when the ultrasound equipment is used, it is possible to treat edema by increasing permeability in blood vessels.

### TECHNICAL FIELD

The present invention relates to an ultrasound equipment for treating edema, and more particularly, to an ultrasound equipment to relieve symptoms of edema or reduce cellular and tissue edemas by generating 20˜400 mW/cm² of low intensity ultrasound and use thereof.

### BACKGROUND ART

Water accounts for 60˜70% of our body weight. Two thirds of the total body water is found within the cells and a third is outside the cells. Edema is an observable swelling from excessive accumulation of body fluids such as plasma, interstitial fluid and transcellular fluid, which is forced out of the blood vessels.

Cellular and tissue edemas develop rapidly following traumatic brain injury or brain ischemia, and the like. They also occur due to a gradual increase in the body fluids such as the increase of synovial fluid by arthritis and the setting of hydrocephalus and glaucoma. If edema is not adequately treated in its early stage, cells and tissues will be severely damaged gradually. There are many methods for treating edema, but they are not efficient and thus it is in desperate need to develop a method for effectively treating edema.

For treating edema, diuretics are mainly used. Diuretics reduce the symptoms of edema by decreasing renal tubular Na⁺, Cl⁻ and water re-absorption to increase excretion of solutes and water, thus eliminating extracellular fluid from the body. However, the prescription of diuretics restricts its use for limited period of time because long-term diuretic use could cause severe side effects such as dehydration, hypothyroidism, hyperadrenalism, brain stroke, renal failure, and the like.

Recently, ultrasound is used for treating various diseases. For example, ultrasound is known as being effective for the delivery of drugs and genes, treating fractures and osteoporosis, thrombolysis (Mitragotri, S., Nature, 4:255, 2005; Zderic, V. et al., Cornea, 23:804, 2004), differentiation of mesenchymal stem cells into chondrocytes (Schumann, D. et al., Biorheology, 43:431, 2006) or osteoblasts (Yang, R. S. et al., Bone, 36:276, 2005) and the like.

However, there has been no report to date that confirms therapeutic effects or symptom-relieving effects of ultrasound on cellular and tissue edema.

Accordingly, the present inventors have made extensive efforts to develop a method for effectively treating cellular and tissue edemas, and as a result, found that when a cellular model and an animal model of edema are continuously treated with ultrasound having an intensity of 20˜400 mW/cm² and a frequency of 0.5˜3 MHz, controlled by a control unit for ultrasound generation, edema symptoms will be relieved and recovered, thereby completing the present invention.

### SUMMARY OF THE INVENTION

Therefore, it is a main object of the present invention to provide an ultrasound equipment for treating edema, which generates ultrasound having an intensity of 20˜400 mW/cm² and a frequency of 0.5˜3 MHz.

To achieve the above object, the present invention provides an ultrasound equipment comprising (a) a control unit for ultrasound generation (130) for generating ultrasound having an intensity of 20˜400 mW/cm² and a frequency of 0.5˜3 MHz; (b) a main control unit (300) for providing a user-set voltage to a probe for a predetermined time; (c) an ultrasound generator (330) for generating ultrasound according to the operating state of the main control; (d) an ultrasound vibrator (340) vibrating according to the value of ultrasound data inputted from the ultrasound generator; and (e) a probe (400) for directly delivering ultrasound energy generated from the ultrasound vibrator to a treatment site, with the probe being in contact with the ultrasound vibrator.

The above and other objects, features and embodiments of the present invention will be more clearly understood from the following detailed description and accompanying claims.

### BRIEF DESCRIPTION OF THE DRAWINGS

FIG. 1 is a prospective view of the ultrasound equipment for treating edema according to an example of the present invention.

FIG. 2 is a flow chart showing an operation of the ultrasound equipment for treating edema using the constitution thereof according to an example of the present invention.

FIG. 3 shows the degree of swelling of erythrocytes treated with gramicidin D, with the passage of time.

FIG. 4 shows changes in cell swelling when erythrocytes, in which edema is induced by treating them with 30 ng/ml of gramicidin D, are treated with ultrasound at various intensities.

FIG. 5 shows the degree of swelling of erythrocytes according to the concentration of mercury chloride while comparing the degree of swelling of erythrocytes suspended in saline solution with that of erythrocytes suspended in hypotonic solution.

FIG. 6 is a graph showing the results of treating erythrocytes, in which aquaporin activity is inhibited by mercury chloride, with ultrasound.

FIG. 7 is a graph showing changes in water content of cerebrum with the passage of time in animal models after brain impact.

FIG. 8 is a graph showing changes in water content of cerebrum after treating the animal model of cerebral edema with ultrasound.

FIG. 9 shows the results of measuring the degree of blood-brain barrier damage of the animal models after ultrasound treatment.

### DESCRIPTION OF REFERENCE NUMERALS

| | |
|---|---|
| 330: ultrasound generator | 300: main control unit |
| 331: ultrasound emitter | 332: frequency divider |
| 333: waveform generator | 334: output amplifier |
| 335: output matching converter | 340: ultrasound vibrator |
| 400: probe | 110: power supply controller |
| 120: time setter | 130: control unit for ultrasound |
| | generation |

### DETAILED DESCRIPTION OF THE INVENTION, AND PREFERRED EMBODIMENTS

In one aspect, the present invention relates to an ultrasound equipment comprising (a) a control unit for ultrasound generation (130) for generating ultrasound having an intensity of 20˜400 mW/cm² and a frequency of 0.5˜3 MHz; (b) a main control unit (300) for providing a user-set voltage to a probe for a predetermined time; (c) an ultrasound generator (330) for generating ultrasound according to the operating state of the main control; (d) an ultrasound vibrator (340) vibrating according to the value of ultrasound data inputted from the ultrasound generator; and (e) a probe (400) for directly delivering ultrasound energy generated from the ultrasound vibrator to a treatment site, with the probe being in contact with the ultrasound vibrator.

In the present invention, the main control unit preferably further comprises a power supply controller (110) or a time setter (120), and the probe preferably has an oval application end. In addition, the control unit for ultrasound generation is preferably configured to generate ultrasound with an intensity of 100˜400 wM/cm².

In the present invention, the ultrasound is preferably generated in continuous mode or in pulse mode.

In one aspect, the present invention also provides a method for relieving or reducing edema wherein ultrasound (at intensity of 20˜400 mW/cm², and a frequency of 0.5˜3 MHz) is applied to the site of swelling.

In the present invention, it is preferable to use said ultrasound equipment.

In the present invention, the edema preferably broadly includes edema that may be induced by an increase of body fluids in a specific tissue, such as brain edema caused by brain injury, brain edema caused by brain ischemia, edema caused by an increase of synovial fluid by arthritis and edema caused by hydrocephalus and glaucoma. However, the whole body edema is excluded.

Hereinafter, the present invention will be illustratively explained referring to the attached drawings.

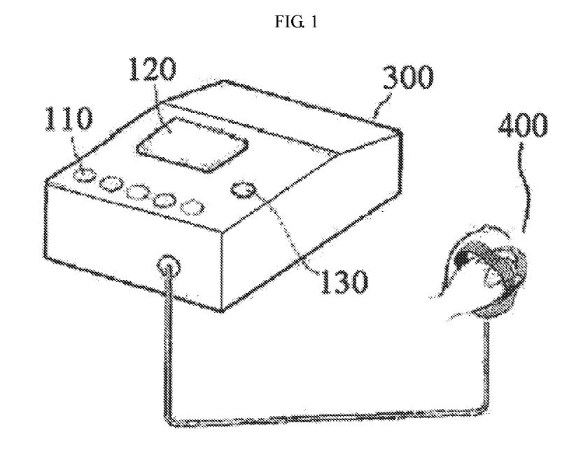

FIG. 1 shows a prospective view of the ultrasound equipment for treating edema according to an example of the present invention. As shown in FIG. 1, the ultrasound equipment in the present invention comprises a main control unit (300) mounted on the outside thereof, and an ultrasound generator (330) and an ultrasound vibrator (340) disposed on the inside thereof, wherein the main control unit is equipped with a power supply controller (110), a time setter (120), a control unit for ultrasound generation (130), and the like in such a way that user can optionally set a power level and an operating time as desired, prior to use of the therapeutic equipment for treating edema. It is preferable that the control unit for ultrasound generation (130) is set to generate ultrasound with an intensity of 80˜100 mW/cm², an optimum condition for edema treatment, which is obtained according to studies by the present inventors.

It is preferable that the inventive ultrasound equipment for treating edema is set in such a way as to be turned off automatically by the time setter disposed in the control unit for ultrasound generation after a predetermined time for edema treatment in consideration of safety since it is developed for individual use at home, and initial set time is preferably approximately 10 minutes, and it is preferably designed such that it can be set to a maximum of 30 minutes.

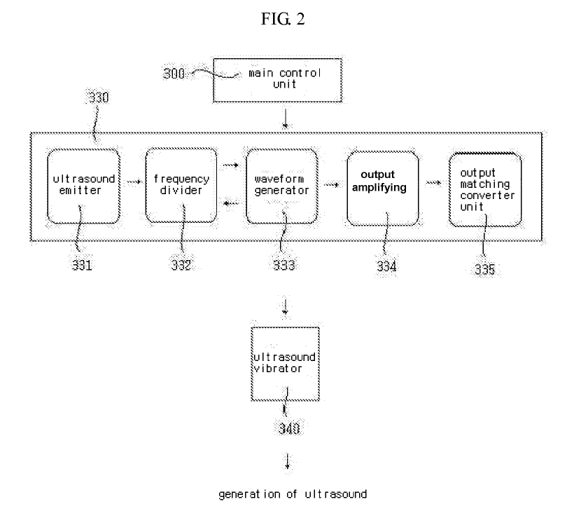

FIG. 2 is a flow chart describing the constitution of the ultrasound equipment for treating edema according to an example of the present invention.

As shown in FIG. 2, the ultrasound generator (330) comprises an ultrasound emitter (331), a frequency divider (332), a waveform generator (333), an output amplifier (334), and an output matching converter (335). By such constitution, the ultrasound is first generated from the ultrasound generator (330) according to the inputted data controlled by a first main control unit, and is subsequently applied to the ultrasound vibrator (340) at the back end thereof. Then, the ultrasound vibrator (340) vibrates the probe in contact with the vibrator itself.

Specifically, the ultrasound emitter (331) receives data controlled by the main control unit to generate basic ultrasound used for generating ultrasound to be finally determined, and the frequency divider (332) receives the waveform of the ultrasound emitter (331) to determine a frequency division according to the state controlled by the main control unit of the therapeutic ultrasound equipment. The waveform generator (333) at the back end thereof receives ultrasound divided in the frequency divider (332) and interrupted and the status of the main control unit (300) to determine a final waveform to be used in the inventive ultrasound equipment for treating edema. The final ultrasound waveform may be in both continuous mode and pulse mode.

The final ultrasound waveform outputted from the waveform generator (333), is applied to the output amplifier (334) on the back end thereof, and the output amplifier (334) amplifies the peak value of the ultrasound waveform determined by a resultant ultrasound waveform operation and power amplification. Then, the amplified waveform outputted from the output amplifier (334) is converted through final output match waveform synthesis, waveform transformation and the like in the output matching converter (335) at the back end thereof. The amplified waveform is finally outputted in a state where it can be applied to the ultrasound vibrator (340) at the back end thereof.

It is preferable that the ultrasound outputted from the ultrasound equipment in the present invention, has 20˜400 mW/cm² of low intensity. In case of treating with a less than 20 mW/cm² of ultrasound, there will be no edema relieving effect, and in case of treating with ultrasound having an intensity of more than 400 mW/cm², there will be a risk of cell damage or cell destruction by ultrasound.

The vibration of the ultrasound vibrator (340) is delivered to the probe having an oval application end to increase cell membrane permeability by a continuous physical force of ultrasound having 20˜400 mW/cm² of low intensity or a biological activity, thus providing the effect of fundamentally treating cellular and tissue edemas.

In order to obtain the preferable effects of the inventive ultrasound equipment for treating edema, the probe may be used in close contact with the site to be treated. It is preferable not to move the probe, once the probe is in close contact with the treatment site, and, after a desired time is set using the time setter (120) of the main control unit, it may be used for less than 30 minutes at a time, 1˜4 times a day.

### EXAMPLES

The present invention will hereinafter be described in further detail by examples. However, it is to be understood that these examples can be modified into other various forms, and the scope of the present invention is not intended to be limited to such examples.

### Example 1

### Effect of Erythrocyte Swelling Reduction

### 1-1: Inducing Erythrocyte Swelling by Gramicidin D Treatment

Ten milliliter of blood was taken from each vein of applicants in their twenties and thirties. The collected blood samples were centrifuged at 3000 rpm for 10 minutes to remove supernatant and buffy coat, and then, the erythrocyte pellets were washed three times with a saline solution (0.9% NaCl).

Gramicidin D is an agent, which allows potassium ions to flow out of the cell and rapidly accumulates nitrite ions in the cell. Thus, erythrocytes treated with gramicidin D becomes swollen due to the difference in ion concentration. In order to induce swelling of erythrocytes, each erythrocyte suspension was treated with 0 ng/ml, 30 ng/ml, 60 ng/ml, 100 ng/ml and 200 ng/ml of gramicidin D, respectively, and centrifuged in a hematocrit centrifuge at 0.5, 1, 2 and 3 hours, thus measuring erythrocyte volume.

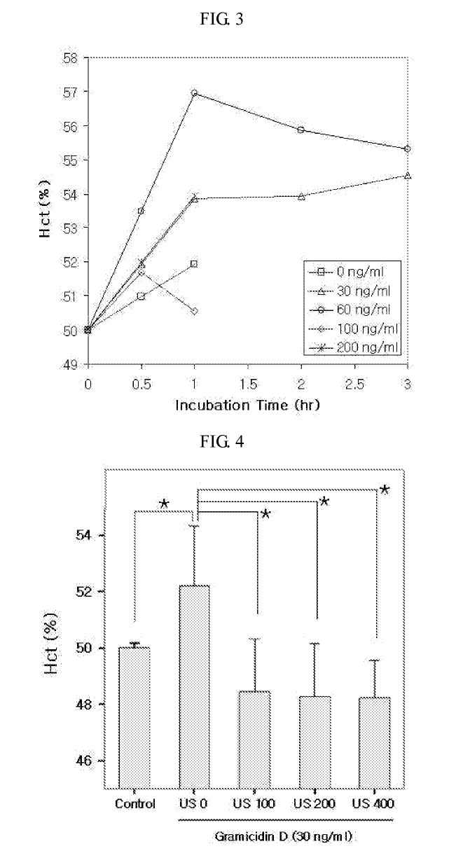

In samples treated with 100 ng/ml and 200 ng/ml of gramicidin D, the erythrocyte volume was increased in its early stage, but, 2˜3 hours after the treatment, the volume could not be measured because of osmotic hemolysis. In a sample treated with 60 ng/ml gramicidin D, erythrocyte volume increased to maximum level at 1 hour after the treatment, and the volume increased by about 7%. In a sample treated with 30 ng/ml gramicidin D didn't show a significant difference compared to the sample treated with 60 ng/ml gramicidin D, but a significant increase in the erythrocyte volume with time was observed (FIG. 3). In order to optimize edema inducement without osmotic hemolysis of erythrocytes, the following experiment was carried out using 30 ng/ml gramicidin D.

### 1-2: Treatment Effect by Ultrasound

Each erythrocyte sample treated with 30 ng/ml gramicidin D to induce edema, which is prepared using the same method described above, was irradiated with ultrasound having intensities of 0, 100, 200, 400 mW/cm² with a frequency of 1 MHz for 20 minutes.

Except for the case where ultrasound intensity was 0 mW/cm², erythrocyte volume was decreased in all samples irradiated with intensities of 100, 200 and 400 mW/cm², and significant differences in erythrocyte volume reduction according to the respective ultrasound intensity were not shown (FIG. 4).

In order to examine osmotic hemolysis of erythrocyte due to ultrasound, erythrocytes were treated with ultrasound to centrifuge at 3000 rpm for 3 minutes, and the supernatant was discarded, followed by a 100-fold dilution, thus measuring at OD 560 nm. When compared with a standard hemolysis curve to determine the hemolysis degree of each sample, no osmotic hemolysis was shown.

It was found that, when erythrocytes, in which edema is induced by treating them with gramicidin D, was treated with ultrasound, edema was decreased without lethal cell damage.

### 1-3: Inhibition of Aquaporin Activity in Erythrocytes

Erythrocytes are known to express Type 1 aquaporin. In order to examine the effect of aquaporins, functioning as a water channel, on edema, erythrocytes were treated with mercury chloride inhibiting aquaporin activity. For the purpose of finding out the optimal concentration at which the volume of erythrocytes can be increased, erythrocytes suspended in hypotonic solution (0.45% NaCl) were treated with mercury chloride ranging from 0.5 μM to 800 μM, and centrifuged in a hematocrit centrifuge 1 hour after the treatment, thus measuring erythrocyte volume.

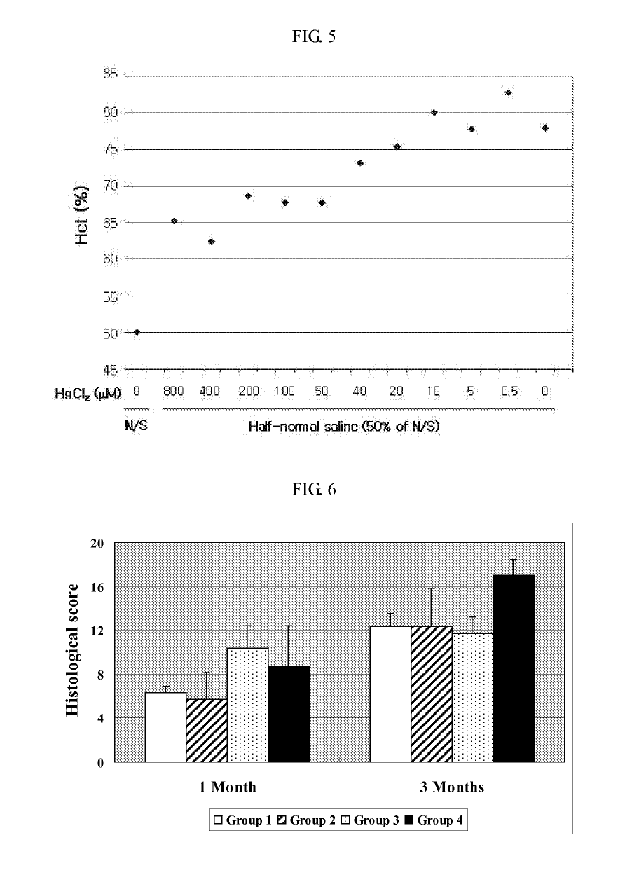

It was shown that the volume of erythrocytes in the hypotonic solution (0.45% NaCl) increased by about 28% compared to erythrocytes in saline solution (0.9% NaCl), and the erythrocyte volume increased as the concentration of the mercury chloride increased. It was observed that there was no concentration-dependent increase when the concentration of mercury chloride was more than 50 μM, and there was no significant difference. Therefore, mercury chloride was used at a concentration of 50 μM to induce swelling of erythrocytes (FIG. 5).

### 1-4: Treatment Effect by Ultrasound

Erythrocytes treated with 50 μM mercury chloride and/or gramicidin D (30 ng/ml), which is prepared by the same method as described in the above example, were irradiated with ultrasound having an intensity of 100 wM/cm² for 20 minutes. As shown in FIG. 6, the sample treated only with gramicidin D showed swelling of erythrocytes (hematocrit 52.7%), compared with the control group, and a decrease in erythrocyte swelling was shown after ultrasound treatment (hematocrit 50.2%). In a group treated only with mercury chloride for comparison showed a little decrease in swelling of erythrocytes, and it was found that, when aquaporin activity was inhibited in erythrocytes in which edema has been induced by treating them with gramicidin D, ultrasound treatment did not contribute to edema reduction (FIG. 6). Therefore, it is suggested that a decrease in erythrocyte edema occurs due to not only a simple physical action but also aquaporin activity.

### Example 2

### Edema Reduction Effect in Animal Models

### 2-1: Animal Models of Edema

In order to establish an animal model of edema, Pentothal (40 mg/kg) was injected into the abdominal cavity of mail Sprague-Dawley rats (270˜330 g) and rats were anesthetized. After anesthesia, the scalp was incised along the midline to expose the surface of the skull. In order to determine the optimal impact condition, a 50 g weight was dropped between bregma and lambda of the skull from various heights of 40 cm, 60 cm, 70 cm, and 80 cm using three rats for each height. As a result, the degree of edema formation was low in groups of 40 cm height and 60 cm height, the degree of edema formation was high in a group of 70 cm height, and rats died immediately after weight drop from a height of more than 80 cm. The animal model of edema was constructed by weight drop from a height of 70 cm using a 50 g weight. A 5-mm craniotomy was performed on the left and right sides of the skull in rats with edema based on sagittal suture using a dental drill (206-103L, Saeshin).

### 2-2: Edema Treatment Effect by Ultrasound

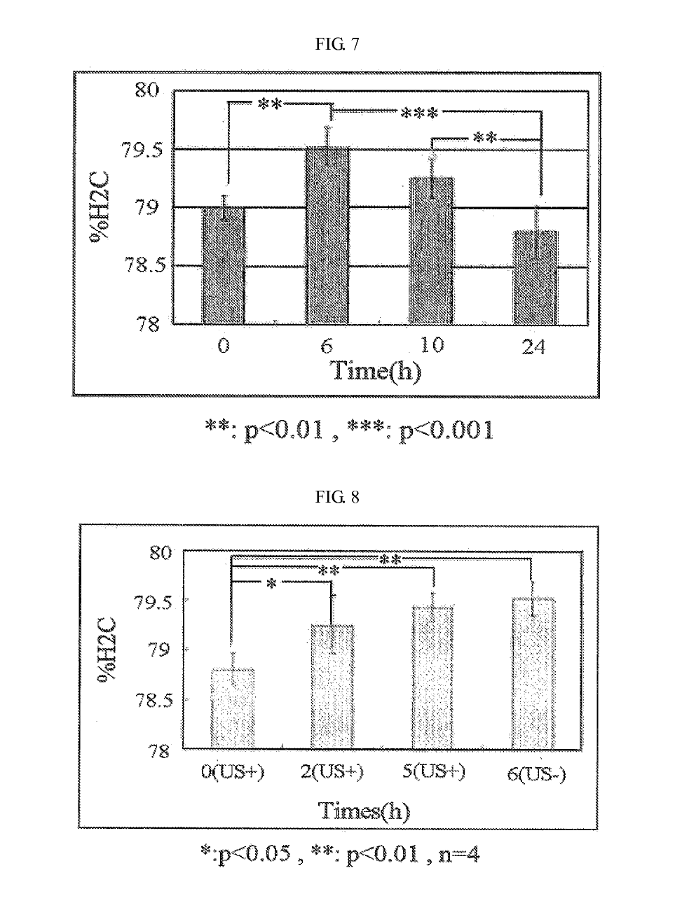

As a result of measuring water content at 0 hour, 6 hours, 10 hours and 24 hours of ultrasound treatment, water content was the highest at 6 hours after impact, and the water content gradually decreased with the passage of time due to dehydration (FIG. 7). In this experiment, ultrasound treatment time was limited to 6 hours, and rats were sacrificed by injecting KC1 (25%, 1 ml) into blood vessels to separate cerebrum. For the measurement of water content, cerebrum weight was measured and the cerebrum was dried at 100 t in a drying oven for 30 hours, and then measured for dry weight thereof. The percentage of water content was calculated by formula, % H₂O=[(wet weight-dry weight)/wet weight]×100

After impact, 5-mm craniotomy was performed on the right and left sides of the skull in rats. Both right and left sides of the skull were treated with ultrasound having at intensity of 100 mW/cm² for 5 minutes and 0 hour, 2 hour, and 5 hour after impact, and then, all rats were sacrificed 6 hours after impact. In the statistical result obtained at 6 hours after impact using a group without ultrasound treatment as a control group, the lowest water content was observed when rats were treated with ultrasound at 0 hour after impact, and the water content increased with time when treated at later time points (FIG. 8).

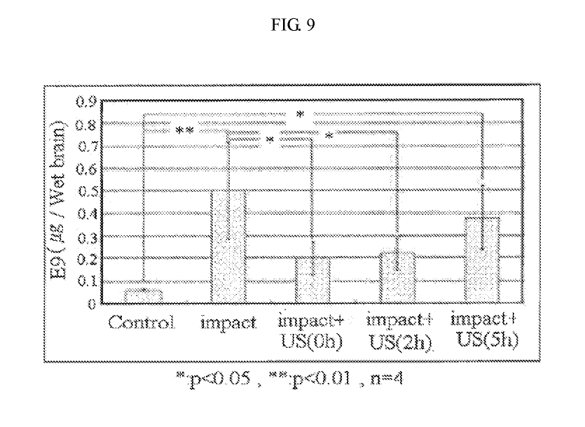

In order to examine the degree of blood brain barrier damage, a 2% (4 ml/kg) Evans blue solution was injected into the femoral vein at 5 hours after impact to extract cerebrum, thus measuring absorbance at 610 nm by ELISA. Blood-brain barrier disruption was not shown at all in normal rats, and the highest value was shown in the sham control group which was not treated with ultrasound. As a result of treating with ultrasound, it was found that the degree of blood-brain barrier damage was low when rats were treated at 0 hour after impact, and the degree of blood-brain barrier recovery decreased with time when treated at later time points (FIG. 9).

### INDUSTRIAL APPLICABILITY

As described above, the ultrasound equipment according to the present invention can be used to relieve or treat cellular and tissue edemas by generating 20˜400 mW/cm² of low intensity ultrasound to increase water permeability of cell membrane or biological membrane, and it can be used easily by simple operation.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

1. An ultrasound equipment for treating edema, which comprises
(a) a control unit for ultrasound generation (130) for generating ultrasound having an intensity of 20˜400 mW/cm² with a frequency of 0.5˜3 MHz;
(b) a main control unit (300) for providing a user-set voltage to a probe for a predetermined time;
(c) an ultrasound generator (330) for generating ultrasound according to the operating state of the main control;
(d) an ultrasound vibrator (340) vibrating according to the value of ultrasound data inputted from the ultrasound generator; and
(e) a probe (400) for directly delivering ultrasound energy generated from the ultrasound vibrator to a treatment site, with the probe being in contact with the ultrasound vibrator.

2. The ultrasound equipment according to claim 1, wherein the ultrasound is generated in continuous mode or in pulse mode.

3. The ultrasound equipment according to claim 1, wherein the edema is selected from the group consisting of brain edema caused by brain injury, brain edema caused by brain ischemia, edema caused by an increase of synovial fluid by arthritis and edema caused by hydrocephalus and glaucoma.

4. A method for relieving or treating edema wherein ultrasound (intensity is 20-400 mW/cm² with a frequency of 0.5-3 MHz) is applied to the site of swelling.

5. (canceled)

6. The method according to claim 4, wherein the edema is selected from the group consisting of brain edema caused by brain injury, brain edema caused by brain ischemia, edema caused by an increase of synovial fluid by arthritis and edema caused by hydrocephalus and glaucoma.

7. A method for relieving or treating edema comprising:
applying the ultrasound equipment of claim 1 to the site of swelling.

8. The ultrasound equipment according to claim 7, wherein the ultrasound is generated in continuous mode or in pulse mode.

9. The ultrasound equipment according to claim 7, wherein the edema is selected from the group consisting of brain edema caused by brain injury, brain edema caused by brain ischemia, edema caused by an increase of synovial by arthritis and edema caused by hydrocephalus and glaucoma.
